(19) United States
(12) Patent Application Publication
(10) Pub. No.: US 2007/0299403 A1
(43) Pub. Date: Dec. 27, 2007

(54) Directional introducer

(76) Inventors: John E. Crowe, Menlo Park, CA (US); Jonathan L. Podmore, San Carlos, CA (US); Michael C. Holzbaur, East Palo Alto, CA (US)

Correspondence Address: SJM/AFD-WILEY, 14901 DEVEAU PLACE, MINNETONKA, MN, 55345-2126, US

(21) Appl. No.: 11/647,295

(22) Filed: Dec. 29, 2006

Related U.S. Application Data
(60) Provisional application No. 60/815,882, filed on Jun. 23, 2006.

(51) Int. Cl. A61M 31/00

(52) U.S. Cl. 604/170.03

(57) ABSTRACT

The invention encompasses various introducer devices, typically used in conjunction with another, elongated medical device. The devices of the invention direct a medical device around or toward a desired anatomical feature through a tube having straight and curved regions. In preferred embodiments, the introducers of the invention are used with a device having a preformed shape, such as the sizing introducers used in atrial fibrillation treatments.

### CROSS-REFERENCE TO RELATED APPLICATIONS

This application claims full priority benefit of U.S. Provisional application 60/815,882, filed Jun. 23, 2006, the entire contents of which are hereby incorporated by reference.

### BACKGROUND OF THE INVENTION

a. Field of the Invention

The invention relates to introducers designed to assist in the placement of catheters, sensors, treatment devices, and/or other medical instruments or devices during a surgical procedure. In a preferred embodiment, a tubular introducer is designed to direct one or more medical devices in or around the pulmonary vein and/or left atrium regions of the heart, especially during an ablation procedure. More particularly, the introducer can be used to straighten and direct the placement of curved measuring elements or ablating elements when used in a modified Cox-Maze procedure, such as a circumferential left atrial ablation around the pulmonary veins. The introducers of the invention advantageously allow users to place one or more curved devices around the pulmonary veins without flexing and holding the curved region into a straightened position.

b. Related Art and Background to the Invention

The use of ablation devices in treating atrial fibrillation (AF) has its roots in the surgical Maze procedure to cure AF developed by Dr. James Cox. The Maze procedure consists of a series of incisions in the right and left atria which effectively isolate the conduction of electrical currents and prevent AF. This approach was patterned on the multiple wavelet theory, where portions of atrial tissue are involved in the development of multiple circuits that can simultaneously or independently maintain the AF condition. The surgical procedure erects blocks in these circuits through scar tissue created by incisions.

One important part of the Maze technique is isolation of the pulmonary veins. In fact, recent observations demonstrate that the initiator circuits of AF typically originate in the pulmonary veins. For many patients, and particularly those with paroxysmal AF, the pulmonary veins are the prime initiating focus of AF.

Currently, techniques to isolate the pulmonary veins can involve the use of devices designed to curve around or encircle the circumference of the pulmonary veins and left atrial tissue. Accordingly, many catheters, introducers, or other devices used have a defined curved region. Placing these devices through a lateral thoracotomy incision can be challenging and time consuming because of the small space in which the surgeon must operate. Thus, there is a need for devices to safely and more efficiently direct these curved devices around an anatomical feature, such as the atrium or pulmonary veins, during the surgical procedure.

### BRIEF SUMMARY OF THE INVENTION

The present invention is directed in part to an introducer device that can be used in conjunction with medical devices, especially those devices having a curved region. In one aspect, a tubular introducer is designed to accept one or more other medical instruments or devices and direct them through a curved exit region at a distal end, in order to position the instrument or device at or around an anatomical feature. In another aspect, the opposite, proximal end of the tubular introducer is designed to be held or gripped by the user while the medical instrument or device is being placed in or around the anatomical feature.

In one embodiment, the invention comprises a tubular device capable of performing a straightener and introducer function, alone or together each function can be particularly useful in performing surgical or minimally invasive surgical procedures, especially where the access to a surgical site involves both a straight and angled insertion, approach, or placement. Thus, in a method to insert a device through the introducer of the invention, part of the insertion may involve a straight direction and another part a curved or angled direction. In another aspect, the “introducer” of the invention essentially refers to a surgical device, cannula, or tube that can be inserted into an incision site to deliver another device and/or allow surgery to be performed at a site by inserting devices through the lumen or interior passageway of the introducer. As explained in more detail below, the introducer is an elongated, hollow tube with at least one distal exit end and proximal end, the distal end designed to be placed at or near the site of surgical intervention or treatment and having a curved region for appropriate placement of inserted devices. The introducer of the invention can also be referred to as a “straightener” in that devices having a curved form or a bias towards a curved form can be inserted and forced into a more linear, straightened configuration.

The design of the tubular introducers of the invention can encompass many options compatible with the devices inserted into them. While the cross sections of the inner and outer surfaces of the introducer are preferably circular, other shapes can also be selected, such as a rectangular inner lumen.

In another aspect, the invention comprises a kit for use in a lateral thoracotomy procedure, the kit including an introducer and one or more instruments or treatment devices. The instruments or treatment devices specifically include a measuring instrument or sizing introducer to measure the circumferential size around a pulmonary vein or left atrial region, and an ablating element or series of ablating elements on a device designed to wrap around the left atrial or pulmonary vein region of heart tissue. The tubular introducer has an interior diameter for the insertion of the one or more instruments or treatment devices, and the shape of the interior passageway can vary according to the shape of the instrument or device. The tubular introducer has a proximal end with one or more contour regions capable of being gripped by the user, and a distal end with a curved exit region to direct the one or more instruments or treatment devices to or at or around an anatomical feature. One or more of the instruments or treatments devices to be inserted into the introducer comprises a flexible curved section that is capable of being partially straightened when introduced into the introducer. A particularly preferred kit comprises an introducer, a sizing introducer or measuring instrument to measure the circumference around an atrial region of the heart, and an ablating device designed to fit around the region measured by the sizing introducer.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is an exemplary introducer capable of being used in a right thoracic approach in an atrial fibrillation treatment.

FIG. 2 is a cross-sectional view of an exemplary introducer.

FIG. 3 depicts a cross-sectional view of a measuring instrument or sizing introducer deployed within an introducer of the invention.

FIG. 4 depicts another cross-sectional view of a sizing introducer deployed within an introducer of the invention.

FIG. 5 through 8 depict cross-sectional views of a sizing introducer being inserted into the introducer of the invention.

FIG. 9 through 12 depict different views of introducers of the invention, showing different angles to direct the introducer toward or around anatomical features, and showing different contours and designs at the gripping end or proximal end.

### DETAILED DESCRIPTION OF THE INVENTION

The headings (such as “Brief Summary”) used are intended only for general organization of topics within the disclosure of the invention and are not intended to limit the disclosure of the invention or any aspect of it. In particular, subject matter disclosed in the “Related Art” includes aspects of technology within the scope of the invention and thus may not constitute solely background art. Subject matter disclosed in the “Brief Summary” is not an exhaustive or complete disclosure of the entire scope of the invention or any particular embodiment.

As used herein, the words “preferred,” “preferentially,” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention and no disclaimer of other embodiments should be inferred from the discussion of a preferred embodiment or a figure showing a preferred embodiment.

In one aspect, the introducer can be used like a cannula to straighten devices as they are being inserted into the body. Such an introducer is generally an elongated, rigid, hollow tube having a proximal (gripping) end, a distal end, and a lumen or passageway extending therebetween. The distal end includes a curved or angled region or region having a defined curvature. In other examples, the introducer can include a shape-memory element or a memory of directionality at the distal end so that it can be partially deformed during use.

A preferred uniform curvature region is used at the angled exit of the tube introducer at the distal end. In one example, the degree of curvature at the distal end is approximated by an osculating circle, meaning a circle that has a common curvature at the point of contact with the distal end. The osculating circle contacts the distal end at the most curved point. The diameter of the osculating circle, D1, is between about two to about three times the diameter of the external diameter, D2, of the lumen or distal end of the introducer. Alternatively, D1 can be greater than or less than D2. More highly curved regions of the distal end can also be selected, as well as regions that have more than one degree of curvature over its length. Alternatively, the curvature at the distal exit end can be defined by an angle of bending. Such angle can be measured by the angle formed at the intersection of a line parallel to the straight proximal region and a line tangent to the curved distal region. The angle can be any suitable angle, and is preferably about 10 to about 70 degrees, and even more preferably about 30 to 65 degrees, or about 40 to 60 degrees.

The introducer can be composed of any biocompatible material or material used in a medical device. Preferably, the material is a polymer that is at least partially transparent, however, stainless steel and other materials can be selected for use. The material selected for the introducer can also be heat-stable, so that the introducer can be sterilized using a suitable sterilization process such as heat sterilization, gamma sterilization, ethylene oxide sterilization or any other suitable sterilization or cleaning process.

Preferably, the introducer has an outer circular cross-sectional shape for smooth insertion into and through tissue. Other shapes can be used such as an oblong, square, rectangular or any other suitable shape. Similarly, the interior shape can be circular in cross-section, oblong, square, rectangular, or any other suitable shape. The inner surface of the lumen or passageway can be custom designed to fit and guide a specific instrument, such as the band-like ablation device as shown in U.S. Pat. No. 7,052,493, which is expressly incorporated herein by reference in its entirety. Such a custom design can also orient the ablating elements in a desired position relative to the tissue it is intended to treat and/or relative to the angle or curvature at the distal end and the anatomical features relevant for the procedure.

The proximal end of the introducer, which typically is not inserted into the body or through tissue, will remain outside of the patient. The shape or contour region at the proximal end can vary. For example, the proximal end can be an ergonomic knob-like shape for the physician to hold onto during a surgical procedure. The proximal end can also include valves and/or ports for liquid and/or other devices entering the introducer, or for irrigation and/or suction. The proximal end can include an indicator, such as on its surface, for indicating the direction of the curve in the distal end. Thus, an indicator can be used as a signal that allows the user to know the direction of the curve at the distal end and the degree to which the introducer can be rotated during a particular procedure to result in a predictable redirection of its distal end. In other embodiments, an indicator can be present on the distal end exit region, such as an electronic device, light, or imaging material that can be viewed on a computer screen.

The distal end of the introducer is closest to the site of use in the patient and will have a curved or directional aspect to at least the exit region. The distal end can be the same shape and cross-sectional diameter as the remainder of the introducer, or alternatively, it can be wider or narrower than other regions of the introducer. Additionally, the distal end can be tapered.

The thickness of the introducer can be uniform and can also be different along the length. For example, the introducer at the proximal end is preferably thicker than at the distal end.

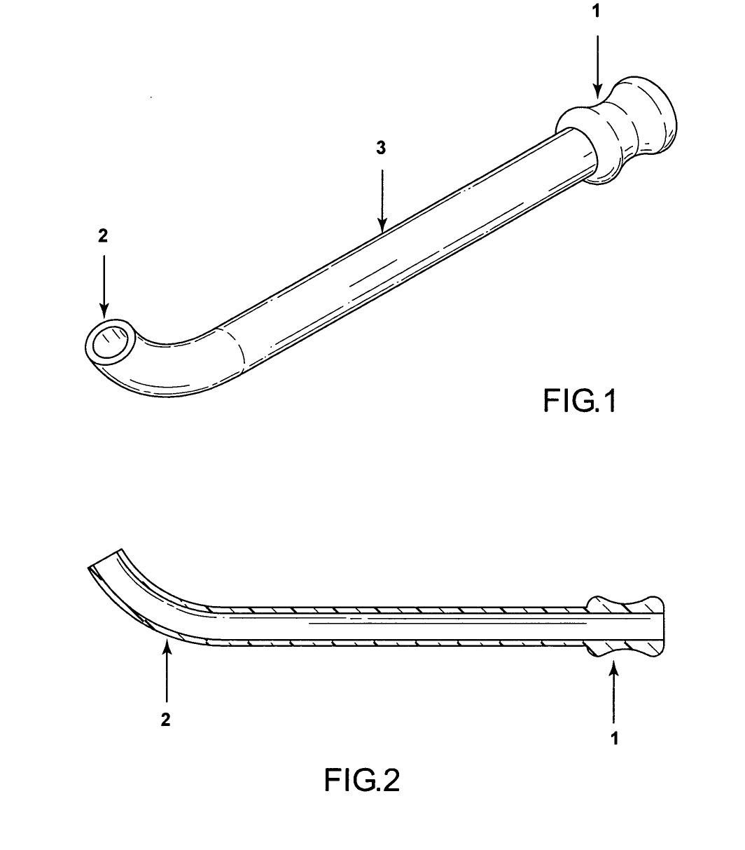

Referring briefly to the drawings, FIG. 1 is an exemplary introducer capable of being used in a right thoracic approach, such as in an atrial fibrillation treatment. The grip end (1) is contoured to be gripped by the user. The distal end (2) is curved to direct a measuring wire and/or a treatment device, such as a catheter, around an atrial region of the heart or pulmonary vein region.

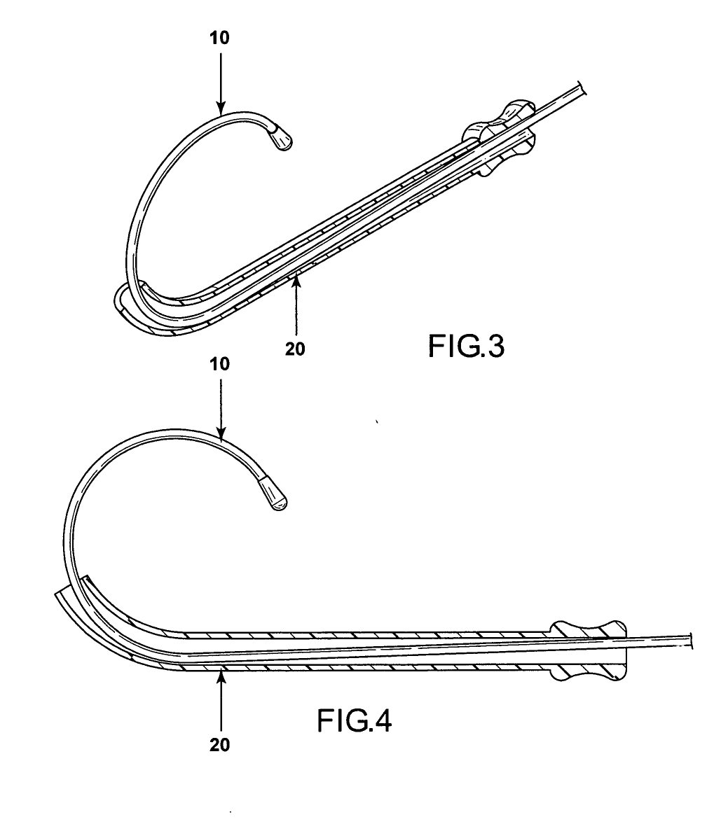

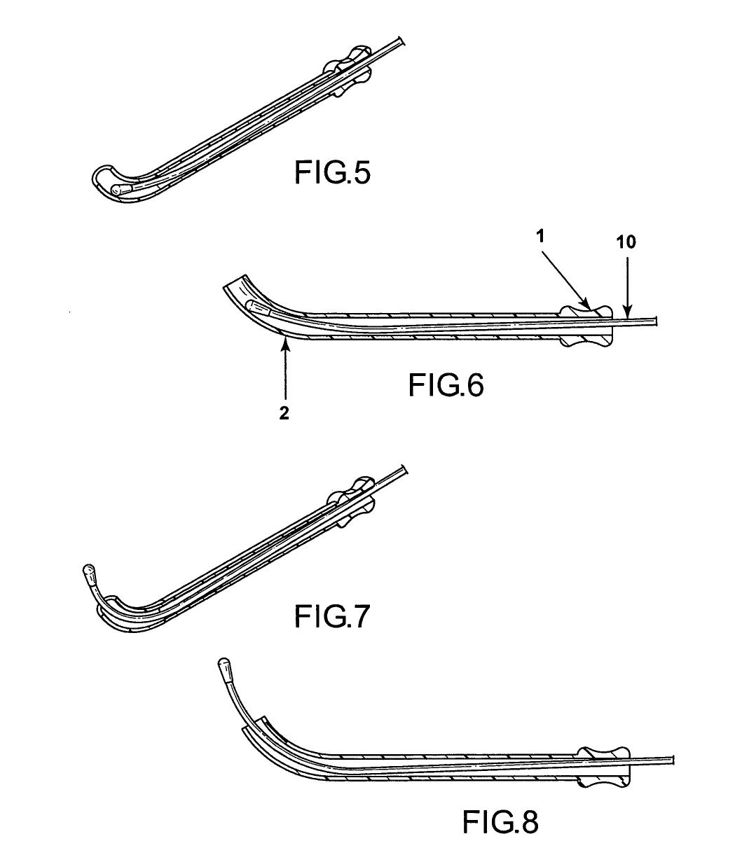

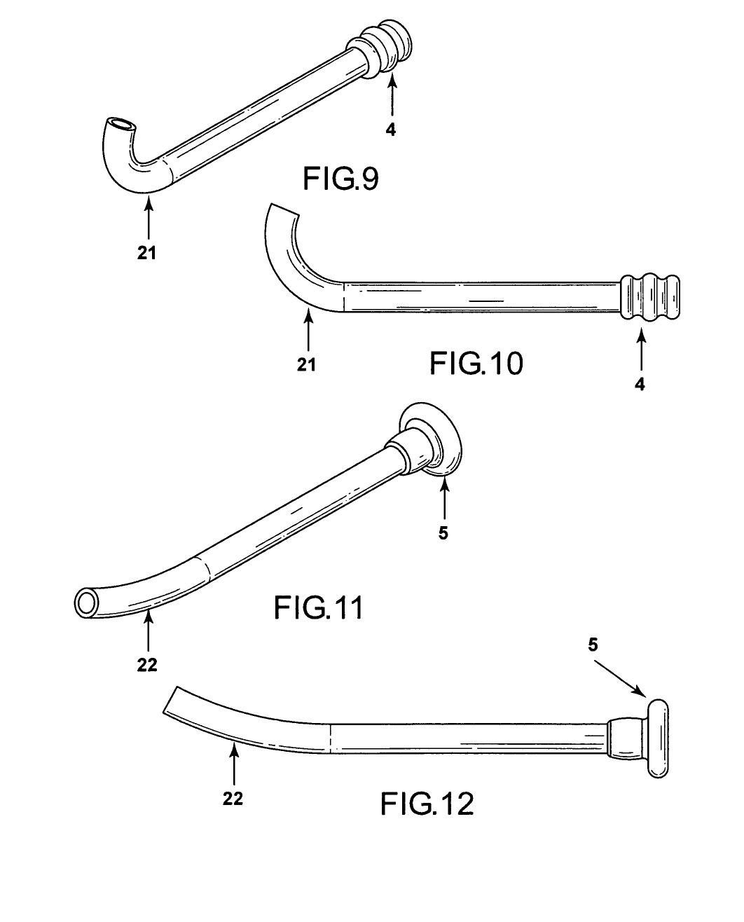

FIG. 2 is a cross-sectional view of an exemplary introducer. FIG. 3 depicts a cross-sectional view of a measuring instrument such as a sizing device (10) deployed within an introducer of the invention, where the measuring instrument is designed with a curved frame or support to direct it around an anatomical feature or region, especially of the heart and/or atrial region of the heart, in order to measure the circumference around an anatomical region. FIG. 4 depicts another cross-sectional view of a curved or shaped device (10) deployed within an introducer (20) of the invention. The curved or shaped device (10) may be a sizing device, an introducer, a catheter, or any other device having a curved or curvable region that is used in a surgical procedure. In the straight area of the introducer (20), the curved or shaped device (10) being fed through the introducer (20) conforms to a straightened shape and can be more easily inserted into a body cavity or around or toward a desired anatomical feature. Also, the curved or shaped device (10) can be more easily manipulated against the rigid passageway of the introducer (20). FIG. 5 through 8 depict cross-sectional views of a curved or shaped device (10) being inserted into the introducer of the invention. The straightening function is evident in FIGS. 5-6. FIG. 9 through 12 depict different views of introducers of the invention, showing different angles (21) and (22) to direct the introducer toward or around anatomical features, and showing different contours and designs (4) and (5) at the gripping end or proximal end of the introducer. The length of the curved region as shown in FIGS. 9-10 can encompass a greater percentage of a circle than as shown in FIGS. 11-12, for example, to direct the instrument on a more circular path.

An instrument or device can be inserted through the passageway of the introducer as shown in FIGS. 3-8. Any instrument can be inserted for the surgery or procedure of interest. However, preferred instruments or devices are the sizing device (10) exemplified in FIGS. 3-8, and the band-like ablation device as shown in U.S. Pat. No. 7,052,493. The sizing introducer and ablation device are manipulated by a surgeon at a proximal end and are then functional at a distal end.

In a preferred aspect and use of the introducer, the introducer is used in an ablation treatment for atrial fibrillation. The introducer is positioned so that an instrument or device is able to curve around the bend at the distal end and therefore be positioned to move around the pulmonary vein in a right thoracic approach to the heart. As shown in FIGS. 3-4, the sizing device inside the introducer can have a curved form or curving-bias form so that it can more easily be positioned around the pulmonary veins and at a desired left atrial placement. This facilitates the measurement of the circumference around a region for ablation. When the desired procedure is finished, the sizing device can be removed from the introducer, which also assists with the more careful removal of the instruments to avoid unintended injury. During removal, the distal end of the sizing device can then curve back through the distal end of the introducer. This process of inserting a sizing device, or any other similar instrument, and removing can be repeated to perform different procedures through the introducer.

Similarly, the ablating device or series of ablating elements can be inserted through the introducer passageway so that it is directed around the pulmonary vein and atrial region in order to cinch or hold the ablating elements at a desired point. The use of the introducer again prevents unintended damage to tissue and facilitates the insertion procedure. The sizing device and the ablating device can be used one after the other by insertion and removal, or the sizing device can be moved out the distal end of the introducer and then followed by the ablating device which is linked, temporarily or otherwise, to the end of the sizing device as it moves through the introducer and then out of the insertion site.

Methods of treatment can be performed by inserting an introducer into a patient, introducing an instrument or device through a passageway of the introducer while maintaining the bend or angle of the distal end of the introducer, utilizing the instrument or device to perform at least one step of a surgical procedure, such as measuring the circumferential size of a left atrial region and/or ablating left atrial tissue, removing the instrument or device from the surgical site and from the introducer, and removing the introducer from the body.

There are many procedures where it may be desirable to use an introducer to be able to change the directionality of the distal end. Thus, a curved distal end of the introducer is advantageous in reaching tissues and structures that are unreachable from a straight insertion and/or present difficulties in bending around tissues through a straight insertion.

Although various embodiments of this invention have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A kit for a lateral thoracotomy procedure comprising an introducer and one or more instruments or treatment devices, wherein the introducer has an interior passageway for the insertion of the one or more instruments or treatment devices, and the introducer has a proximal end with one or more contour regions capable of being gripped by the user, a distal end with a curved exit region to direct the one or more instruments or treatment devices, and a substantially straight region between the proximal end and distal end, and wherein at least one of the one or more instruments or treatment devices comprises a flexible curved section that is capable of being partially straightened when introduced into the introducer.

2. The kit of claim 1, wherein the kit comprises a measuring instrument, designed to fit around an atrial region of the heart, wherein the measuring instrument has a preformed curved region.

3. The kit of claim 2, wherein the kit further comprises a treatment device having one or more ablation elements.

4. The kit of claim 3, wherein the ablation elements comprise an ultrasound ablation element.

5. The kit of claim 1, wherein the curved exit region has a uniform curvature.

6. The kit of claim 5, wherein the curvature is defined by a circle having a diameter of about two times the diameter of the exterior size of the introducer distal end.

7. The kit of claim 5, wherein the curvature is defined by a circle having a diameter of more than two times the diameter of the exterior size of the introducer distal end.

8. An introducer for use in directing a medical instrument or treatment device around an atrial region of the heart, the introducer comprising a curved distal exit end, a proximal gripping end, and a passageway in between, wherein the angle that the curved distal exit end is bent from the plane formed through the axis of the passageway at a straight proximal region of the introducer is between about 10 and about 90 degrees.

9. The introducer of claim 8, wherein the angle is between about 30 to about 65 degrees.

10. The introducer of claim 9, wherein the angle is between about 40 to about 60 degrees.

11. The introducer of claim 8, wherein the proximal gripping end comprises a raised or textured area for gripping by hand.

12. The introducer of claim 8, wherein the introducer is at least partially transparent.

13. The introducer of claim 8, wherein the length of the curved section is designed to direct an instrument or treatment device inserted through the passageway of the introducer around the pulmonary vein region near the left atrium of a human heart.

14. The introducer of claim 9, wherein the length of the curved section is designed to direct an instrument or treatment device inserted through the passageway around the pulmonary vein region near the left atrium of a human heart.

15. The introducer of claim 10, wherein the length of the curved section is designed to direct an instrument or treatment device inserted through the passageway around the pulmonary vein region near the left atrium of a human heart.

16. The introducer of claim 8, wherein the curvature at the curved distal exit end is defined by a circle having a diameter of about two times the diameter of the exterior size of the introducer distal exit end.

17. The introducer of claim 16, wherein the curvature is defined by a circle having a diameter of more than two times the diameter of the exterior size of the introducer distal exit end.

18. The introducer of claim 8, further comprising at least one port at the proximal end of the introducer.
